(19) United States
(12) Patent Application Publication
(10) Pub. No.: US 2009/0299292 A1
(43) Pub. Date: Dec. 3, 2009

(54) PERCUTANEOUS GASTROSTOMY CATHETER INCLUDING A SINGLE BIODEGRADABLE INTERNAL FLANGE

(76) Inventor: Serge Renaux, Lamalou les Bains (FR)

Correspondence Address: JEROME D. JACKSON (JACKSON PATENT LAW OFFICE), 211 N. UNION STREET, SUITE 100, ALEXANDRIA, VA, 22314, US

(21) Appl. No.: 12/375,019

(22) PCT Filed: Jul. 31, 2007
(86) PCT No.: PCT/FR2007/001320

§ 371 Date: May 13, 2009

(30) Foreign Application Priority Data
Aug. 2, 2006 (FR) 0607074

(51) Int. Cl. A61M 25/04

(52) U.S. Cl. 604/175

(57) ABSTRACT

The invention relates to a percutaneous gastrostomy probe including a tube (1), an internal retaining means (2) and an external flange (3), essentially characterised in that the internal retaining means is formed by a single flange (2) made entirely from a biodegradable copolymer The type of polymers used, the dose and molar mass in number thereof and the thickness of the flange are determined so as to produce (i) a retaining means that combines flexibility and elastomer properties and (ii) a connection between the flange (2) and the tube (1), having a tearing threshold that is a function of the stoma creation time.

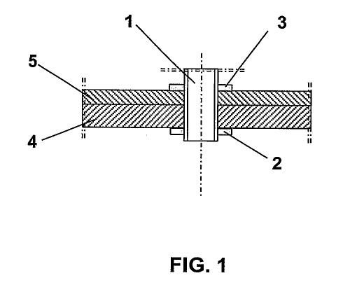

### FIELD OF INVENTION

The present invention relates to percutaneous endoscopic gastrostomy (PEG) and radiological percutaneous gastronomy (RPG) catheters enabling direct access to the gastric cavity for enteral feeding.

### BACKGROUND TECHNOLOGY

Percutaneous gastrostomy is currently the standard route for prolonged enteral feeding. Due to the simplicity and speed of technology and the development of the equipment, gastroenterologists or radiologists are increasingly sought, and the placement is accessible to any endoscopist or radiologist.

There are two placement techniques:
the endoscopic <<pull>> technique, mainly used by gastroenterologists: the steril PEG kits generally include a puncture trocar, a double strand of wire, a tubular gastrostomy catheter, a flange-type internal means of retention, an external fixation flange: ideally, crocodile or rat tooth grasping forceps are used to grasp the intragastric wire. A diathermic cove or biopsy forceps can also be used;
the <<push>> technique via the abdominal wall, used mainly by radiologists: in this case anchors enable binding of the stomach to the abdominal wall for the time required for the formation of adhesions between the external part of the stomach and the abdominal wall. A catheter balloon is then positioned through the abdominal wall using a dilator and a peel-off nozzle.

The catheters are generally made of silicone or polyurethane, inert and well tolerated materials. Various sizes or Charrières are available, the small catheters being blocked more easily.

There are extractable and non-extractable catheters.

Non-extractable catheters, to be replaced, should be cut flush with the cutaneous orifice. The internal device is then pushed into the stomach.

The internal means of retention can be retrieved by endoscopy, an operation that can turn out to be delicate, or evacuated via the natural routes with risks of obstruction and intestinal perforation.

The advantage of non-extractable catheters lies in their relatively rigid internal flange thus resisting an attempt of pulling out by an agitated restless patient.

The extractable catheters have a retractable removable internal flange, or a deflatable retention system, enabling their removal via the cutaneous orifice by firm pulling.

The advantage of these flexible systems is to be able to pass through a severe stenosis, avoiding endoscopy, but they have less resistance to pulling out.

The choice between extractable and non-extractable catheters, made of silicone or polyurethane, depends on the indication, taking into account the advantages and disadvantages of each type of catheters.

The extractable catheters made of silicone are suitable for temporary enteral feeding.

Non-extractable or polyurethane catheters are more suitable for permanent enteral feeding or a restless patient.

The replacement of the gastrostomy catheters may be needed in case of obstruction, tube deterioration (cracking, porosity, expansion, colonization by candida).

The majority of replacement devices are catheters with a water-inflatable balloon, made of silicone. Their suitability for the gastric environment and an external retention flange enables safe use.

There is also a shorter gastrostomy button and on bare skin which, because of its aesthetic advantage and comfort, is indicated in the young or outpatient subject. It cannot be installed initially except with difficulty, however, and comes most often as replacement for a catheter already positioned in the stomach.

The catheter according to the invention is the type including:

a) a tubing designed to pass through the stomach and abdominal walls of the subject,

b) a flange-type internal means of retention designed to be joined to the aforementioned tubing and to be maintained pressed against the inner face of the stomach wall;

c) an external flange, traversed by the aforementioned tubing, designed to be pressed against the external face of the abdominal wall and exert, in cooperation with the internal means of retention, a pressure adapted to press the stomach wall against the abdominal wall in the area of the stoma.

### SUMMARY OF INVENTION

The invention aims to realize a catheter of the type in question, designed to implement a new and original solution to eliminate the drawbacks mentioned above.

To that end, it relates to a percutaneous endoscopic gastrostomy catheter, which is essentially characterized in that the internal means of retention is constituted by a single flange made entirely of a biodegradable copolymer, of which the nature of polymers used, their amount, and their molar mass number, as well as the thickness of the aforementioned flange, are determined to obtain:
first, a means of retention that combines the properties of flexibility facilitating its endoscopic installation and elastomers enabling it to resume its original shape when it is pressed against the stomach wall;
second, a flange and tubing connection that has a pulling breakage threshold that is a function of the formation time of the stoma.

The copolymer used is optimally chosen among the triblocks PLA-PEG-PLA and PLA GA-PEG-PLA GA.

The biodegradation of internal flanges constitutes one of the advantages of the invention, eliminating:
the risks of occlusion with fatal consequences;
the use of an endoscope, which requires anesthesia.

The period of optimal healing of the stoma is approximately 21 days. Too short a period could lead to its poor healing with all the consequences that may arise mainly at the introduction of a replacement catheter.

The incidents resulting from this mainly relate to the incarceration of the flange between the walls, or the accidental passage of the administered nutritious products into the abdominal cavity. These incidents appear to be related to the removal by force of the gastrostomy tube through the stoma, leading to the disengagement of the flange and causing laceration of the stoma. The positioning of the replacement catheter in this case is uncertain.

### PRESENTATION OF FIGURES

The characteristics and advantages of the invention will emerge more clearly upon reading the following detailed description of at least one preferred implementation thereof given by way of non-limiting example and illustrated in the attached drawing (single FIGURE) which sectionally represents a partial view of a catheter provided with a single internal flange, sandwiching the stomach and abdominal walls.

### DETAILED DESCRIPTION OF THE INVENTION

The illustrated percutaneous gastrostomy catheter is the type including:
a tubing (1) designed to pass through the stomach (4) and abdominal (5) walls of the subject;
an internal means of retention (2) designed to be joined to the tubing (1) and to be maintained pressed against the internal face of the aforementioned stomach wall (4);
an external flange (3), traversed by the tubing (1), designed to be pressed against the external face of the aforementioned abdominal wall (5) and exert, in cooperation with the internal means of retention (2), a pressure adapted to press the stomach wall (4) against the abdominal wall (5) in the area of the stoma.

The internal means of retention is constituted by a single flange (2) made entirely of a biodegradable copolymer, of which the nature of polymers used, their amount, and their molar mass number, as well as the thickness of the aforementioned flange, are determined to obtain:
first, a means of retention that combines the properties of flexibility facilitating its endoscopic installation and elastomers enabling it to resume its original shape when it is pressed against the stomach wall;
second, a flange (2) and tubing (1) connection that has a pulling breakage threshold that is a function of the formation time of the stoma.

The speed of biodegradation of the means of retention is programmed so that its mechanical characteristics are maintained at least until the adhesion of the stomach and abdominal wall between themselves.

The flange (2), which has an opening for the passage of the tubing (1), may be joined to it by gluing, welding, crimping or by any other known means.

The tubing (1) is generally made of a non-biodegradable biocompatible material such as, for example, silicone or polyurethane.

The connection of the internal retention flange (2) to the pipe (1) is designed to disengage itself from the latter under a well-defined external traction on it.

The choice of biodegradable copolymer or polymer suitable for the considered medical application was the subject of tests including synthesizing various polymers and copolymers into samples, immersed in a model of gastric fluid, with dimensions similar to those of the biodegradable internal means of retention concerned, capable of degrading within a well termined period (in particular between 1 and 3 months), having the physical and mechanical characteristics required, in terms of hardness or elasticity, shape changes, swelling due to the water, decomposition.

Tests were conducted on samples made with various polymers or copolymers of the types:
Polylactic acid such as PLA 50 (Mₙ=21000 g/mol) and PLA 50 (Mₙ=46000 g/mol);
Polylactic acid-glycolic acid such as PLA 37.5-GA 25(Mₙ=39000 g/mol);
Triblocks PLA-PEG(poly ethylene glycol)-PLA such as PLA 50-PEG(20000)-PLA 50 (Mₙ=277760 g/mol), PLA 50 -PEG (20000)-PLA50 (Mₙ=100600 g/mol), PLA 50 -PEG(6000)-PLA50(Mₙ=56400 g/mol), PLA 96-PEG 12000-PLA 96 (Mₙ=68311 g/mol), PLA 96-PEG 8000-PLA 96 (Mₙ=71684 g/mol) and PLA GA-PEG-PLA GA PLA.

The choice was made in the PLA 96-PEG 12000-PLA 96 (Mₙ=68311 g/mol), PLA 96-PEG 8000-PLA 96 (Mₙ=71684 g/mol) and PLA GA-PEG-PLA GA, class of copolymers, to implement the internal flange, which must conform to the following criteria:
a higher PLA (L) amount, above 60, to obtain the best compromise between the elastomer qualities (shape memory) and flexibility;
an appropriate amount of molar mass (Mₙ) of the PEG for better management of degradation time;
appropriate dimensions of the flange: thickness optimally between 1 and 3 mm because it also affects the speed of biodegradability.

Components having the effect of changing the mechanical characteristics and the rate of degradation as well as body tolerance, can be added to the aforementioned copolymers.

Of course, the invention is not limited to the implementation modes described and illustrated for which the person of skill in the art will be able to provide other variations, particularly in the types of catheters employed and the materials constituting the subassemblies of the aforementioned catheters provided that the internal flange is completely biodegradable.

1-4. (canceled)

5. A percutaneous gastrostomy catheter, including:
a) a tubing configured to pass through the stomach and abdominal walls of the subject;
b) an internal means of retention configured to be joined to the tubing and to be maintained pressed against the inner face of the stomach wall;
c) an external flange, traversed by the tubing configured to be pressed against the external face of the abdominal wall and exert in cooperation with the internal means of retention, a pressure adapted to press the stomach wall against the abdominal wall in the area of the stoma,
characterized in that the internal means of retention includes a single flange made entirely of a biodegradable copolymer including a PLA-PEG-PLA triblock having, first, flexibility properties facilitating its endoscopic installation and elastomeric properties enabling it to resume its original shape when it is pressed against the stomach wall and, second, a flange and tubing connection having a pulling breakage threshold that is a function of the formation time of the stoma.

6. A catheter according to claim 5 wherein the copolymer includes a PLA GA-PEG-PLA GA triblock.

7. A catheter according to claim 5 wherein the copolymer includes the PLA 96-PEG 12000-PLA 96 (Mₙ=68311 g/mol) triblock.

8. A catheter according to claim 5 wherein the copolymer includes the PLA 96-PEG 8000-PLA 96 (Mₙ=71684 g/mol) triblock.

9. A catheter according to claim 5 wherein the flange has a thickness of between 1 and 3 mm.
